(19) United States
(12) Patent Application Publication
(10) Pub. No.: US 2017/0071605 A1
(43) Pub. Date: Mar. 16, 2017

(54) ABSORBABLE TISSUE RECONSTRUCTION DEVICE, IN PARTICULAR FOR TISSUES SUCH AS LIGAMENTS

(71) Applicant: ANTONIO SAMBUSSETI, CORSO DI PORTA VITTORIA 9 (IT)

(72) Inventor: Antonio SAMBUSSETI, Cremona (IT)

(21) Appl. No.: 15/124,603

(22) PCT Filed: Mar. 11, 2015
(86) PCT No.: PCT/EP2015/055045

§ 371 Date: Sep. 8, 2016

(30) Foreign Application Priority Data
Mar. 11, 2014 (IT) MI2014A000383

(51) Int. Cl. A61B 17/11

(52) U.S. Cl. CPC A61B 17/1146; A61B 2017/00004

(57) ABSTRACT

There is described an absorbable device (1) for the connection and reconstruction of a ligament, comprising a central reconstruction portion (9) and two connecting 5 ends (10a, 10b) at the two ends of said central portion (9), characterized in that in said connecting device, said two connecting ends (10a, 10b) comprise two retaining portions (11a, 11b), respectively, for receiving fixing means adapted to fix said device (1) to said ligament.

### BACKGROUND ART

Ligaments are formations of fibrous connective tissue and combine two or more anatomical structures. The cruciate ligaments of the knee joint are fibrous bundles crossed with one another to support the joint itself and allow its joint movements. Many individuals—in particular those who practice a sport—undergo damage to these ligaments. These ligaments are not the only ones to get damaged, however they are the most frequent and most critical ones as regards their recovery process.

In order to reconstruct the tendons damaged upon a lesion there exists a technique known as “autologous”. According to this surgical technique, a fiber bundle is used, which is taken from the ligaments which are used as a basis to reconstruct the damaged ligament. This technique is very invasive and cannot be always applied.

In response to the “autologous” technique, devices have been developed to “simulate” a ligament. Such devices have the shape and size of the ligament to be replaced and are made of polyester. At the ends thereof, there are fixing means (staples, anchors or absorbable or non-absorbable synthesis screws) to stably fix the artificial ligament and allow the partial reconstruction of the ligament tissue thereon. The implant of artificial ligaments of this type is final, over time a part of ligament reconstructs thereon and integrates its functions. Such devices ensure a good biocompatibility, however they have some drawbacks related to their mechanical function. In fact, their flexibility is acceptable but not excellent. Therefore, the performance of such devices is valid even if with some drawbacks, especially as regards the mechanical functionality. However, such a drawback can be especially insidious because such devices can be damaged even shortly after their installation, thus requiring a new operation to replace the device.

These drawbacks caused the “autologous” technique to be the predominant choice despite its limitations.

### SUMMARY

Therefore, the known techniques described do not allow to have a device for the connection and reconstruction of torn ligaments.

This situation is particularly disadvantageous both for patients who suffer from such injuries and for the practitioners who apply non-optimal solutions to the problem in place, which often requires later corrective interventions or cause further damage to the patient.

It is the general object of the present invention to implement a device of the reversible type for the connection and reconstruction of a ligament, which allows the problems of the prior art to be solved in a simple and cost-effective manner.

It is the first specific object of the present invention to implement a device of the reversible type for the connection and reconstruction of a ligament, which is applicable irrespective of the physiological conditions of the individual to whom it is intended.

In general, a device for the reconstruction and connection of a ligament according to the present invention comprises a central reconstruction portion and two connecting ends, at the two ends of the central portion, characterized in that said two connecting ends are shaped to receive fixing means adapted to fix the device to said ligament.

Further advantageous technical features of the device according to the present invention are described in the dependent claims.

### LIST OF FIGURES

The technical features of the present invention as well as advantages thereof will become apparent from the following description to be considered in conjunction with the accompanying drawings, in which:

FIG. 1 shows a top view of a tissue reconstruction device according to the present invention;

FIG. 2 shows a sectional view of a second embodiment of a tissue device according to the present invention.

### DETAILED DESCRIPTION

The following description and the accompanying drawings are intended for illustrative purposes and therefore they do not limit the present invention, which may be implemented according to other and different embodiments; moreover, it is worth noting that these figures are diagrammatic and simplified.

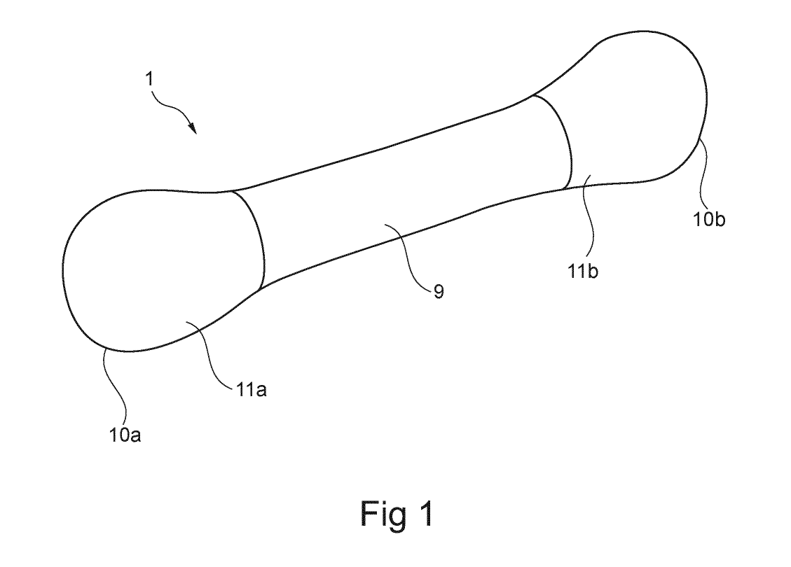

With reference to FIG. 1, there is shown a device of the absorbable type for the connection and reconstruction of ligaments according to the present invention. The term “absorbable” means a device to be installed at a damaged ligament to allow the physiological tissue reconstruction thereof. The device is absorbed by the tissues together with the tissue reconstruction after a predetermined period of time.

Such a device can be applied to a single ligament (e.g. the anterior cruciate ligament of the knee joint) or to a group of ligaments (e.g. the rotator cuff of the shoulder).

Device 1 comprises a central reconstruction portion 9 and two connecting ends 10a, 10b at the two ends of the central portion 9. The two connecting ends 10a, 10b comprise two retaining portions 11a, 11b respectively, for receiving fixing means adapted to fix the device 1 to the ligament.

It is worth noting that the ends 10a and 10b have a general stiffness in order to receive the fixing means which is greater than that of the central portion 9. Thereby, said fixing means, known per se, which comprise either absorbable or non-absorbable staples, anchors or synthesis screws, are applied to the ends 10a and 10b. Their configuration, in particular their stiffness, allows the fixing means to be retained stably until they are completely physiologically reabsorbed. This situation is particularly advantageous: the fixing means stably retained until their reabsorption ensure that device 1 is in turn in a predetermined position until it is reabsorbed. A proper tissue reconstruction of the ligament for which device 1 is intended is thus ensured.

The tissue reconstruction of the ligament for which device 1 is intended takes place on the central portion 9 and on ends 10a and 10b.

In use, the device is placed in a predetermined reconstruction position according to anatomical constraints and to the extent of the ligament portion to be reconstructed. It is therefore essential, for a successful reconstruction of the ligament, that the device remains in such a position for the entire duration of the reconstruction process.

To this end, the two connecting portions 10a, 10b are configured to receive the fixing means stably after the operations aimed to place it in its reconstruction position. In this first condition, the device has such a structure as to favor the tissue regrowth.

Thereafter, the physiological reconstruction process absorbs the fixing means and the device 1 itself. The ligament (or ligaments in the case of a bundle of ligaments for which a device according to the present invention is to be used) under reconstruction has its tissue regrowth from the ends 10a and 10b to continue then on the central portion 9 of the device.

In this second condition, the device has such a structure as to support the tissue regrowth, and in particular up to its complete reconstruction.

According to a preferred embodiment, device 1, i.e. the central portion 9, the connecting ends 10a, 10b and the two connecting portions 11a and 11b are made of polyglycolic acid (also known as PGA).

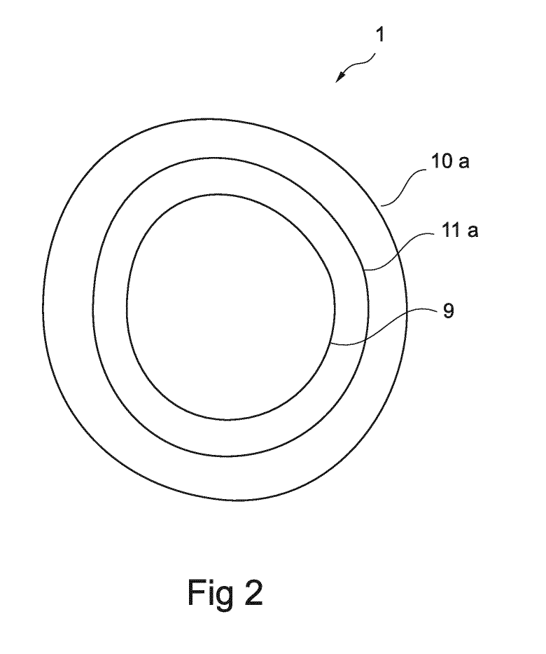

As seen in FIG. 2, according to an embodiment of the device, the two connecting ends of 10a, 10b have a cross-section which is greater than that of the central portion 9.

According to a further embodiment (shown in FIG. 1), the connecting ends have a rounded shape.

Preferably, the central portion 9 of a device according to the present invention is a circular-base prism. Of course, different shapes are suitable to implement the section of the central portion 9, they may be evaluated from time to time in the light of the structural constraints to be met.

According to a further embodiment, the central portion 9 is a hollow cylinder. However, according to an alternative embodiment, the central portion 9 is a solid cylinder.

The dimensions of the central portion 9 are devised to fit any size of ligaments for which the device is intended. Such a portion has a diameter from 1 to 15 mm; a length from 5 to 45 mm.

Moreover, the connecting portions 10a, 10b are fixed to the central portion, respectively, overlapping by at least 1.5 cm said central portion 9.

1. An absorbable device (1) for the connection and reconstruction of a ligament, comprising a central reconstruction portion (9) and two connecting ends (10a, 10b) at the two ends of said central portion (9), characterized in that said two connecting ends (10a, 10b) comprise two retaining portions (11a, 11b), respectively, for receiving fixing means adapted to fix said device (1) to said ligament.

2. A device according to claim 1, wherein said central portion (9), said two connecting ends (10a, 10b) and said two retaining portions (11a, 11b) are made of polyglycolic acid.

3. A device according to any of the preceding claims, wherein said two connecting ends (10a, 10b) have cross-section greater than said central portion (9).

4. A device according to any of the preceding claims, wherein said two connecting ends (10a, 10b) have a rounded shape.

5. A device according to any one of the preceding claims, wherein said central portion (9) is a circular-base prism.

6. A device according to claims 1 to 5, wherein said central portion (9) is a hollow cylinder.

7. A device according to any one of the preceding claims, wherein said central portion (9) has a diameter in the range from 1 to 15 mm.

8. A device according to any one of the preceding claims, wherein said central portion (9) has a length in the range from 5 to 45 mm.
